(19) United States
(12) Patent Application Publication
(10) Pub. No.: US 2012/0157859 A1
(43) Pub. Date: Jun. 21, 2012

(54) DEVICE TO ASSIST IN THE PRACTICE OF A PHYSICAL ACTIVITY SESSION, AND PHYSICAL ACTIVITY APPARATUS PROVIDED WITH SUCH A DEVICE

(75) Inventor: Bernard Hecquet, Lille (FR)

(73) Assignee: SOCIETE DE RESSOURCES ET DE DEVELOPPEMENT POUR LES ENTREPRISE ET LES PARTICULIERS SRDEP, Lille (FR)

(21) Appl. No.: 13/382,939

(22) PCT Filed: Jul. 8, 2010
(86) PCT No.: PCT/FR2010/000502

§ 371 Date: Mar. 13, 2012

(30) Foreign Application Priority Data
Jul. 9, 2009 (FR) 09/03.394

(51) Int. Cl. A61B 5/0205

(52) U.S. Cl. 600/484

(57) ABSTRACT

The invention relates to a device to assist in the practice of a physical activity session by an person. The device has an electronic means for determining the real-time physical effort to be provided by said person during said session, implementing a control law having, as an output, a parameter that corresponds to the physical effort to be provided (EPF) and, as an input, data collected while the force is being applied, essentially comprising: the measurement of the heart rate (FC) of said person; the measurement of the respiratory rate (FR) of said person; and one or more indices (EVA) of strenuousness of the effort as perceived by said person, to be specified by said person during the effort, and in which said control law depends essentially on respective programmed thresholds which should not be exceeded, essentially comprising: the maximum heart rate (FC_{M}), the maximum respiratory rate (FR_{M}), and the maximum value or values associated with said index or indices of strenuousness.

The invention relates to a device for assisting in the practice of a physical activity, and a physical activity apparatus equipped with such a device.

The invention can particularly be applied to enable the practice of physical exercise by fragile subjects for whom this practice is liable to involve health risks, by limiting said risks.

In the field of sport, physical activity apparatuses are known, such as exercise bikes, treadmills, rowing machines, which are routinely used in gyms or for sports physicals.

Moreover, sports physicals are useful for detecting contraindications for practicing sport, and also for checking fitness levels with a view to improving a sportsperson's physical condition.

Frequently, these tests are based on the measurement of a physiological parameter (heart rate, or breathing-related parameter) and require the use of a measurement apparatus such as a cardio frequency meter, electrocardiogram, or others.

Some of these tests are delicate and should only be performed under medical supervision.

That being said, in the case of fragile subjects, the practice of physical activity may be recommended, but involves a risk, however. The main risk is sudden cardiac arrest.

At the present time, to the inventor's knowledge, there is no specific tool for supporting a fragile subject in the practice of a physical activity so as to limit the risks thereof.

However, the prior art discloses, from the document US 2005/0070809, a system for regulating a user's physical exercise.

In this system, the level of instantaneous exertion is compared to a target exertion and the user is guided by the system to increase, decrease or maintain his/her exertion.

The instantaneous exertion is measured by means of various input parameters which are collected by handheld electronics.

According to the example developed in the document US 2005/0070809, the input parameter is the heart rate. Further parameters may be collected, such as the body temperature, respiratory rate, air temperature, air humidity, or travelling speed (running).

It is noted that all these parameters are “objective” data measured scientifically by various sensors.

The control law implemented in the system according to the document US 2005/0070809 thus determines, in real time, the exertion to be provided by the user only on the basis of these objective parameters, which do not account directly for the user's perceptions (such as pain, fatigue, etc.).

The principle of the present invention is that of proposing a device for assisting in the practice of a physical activity session wherein the control law is not only dependent on objective parameters collected by various sensors such as those known from the prior art, but also on subjective parameters—indices of strenuousness of the exertion—specified by said subject during the exertion.

The aim of the present invention is thus that of proposing a device for assisting in the practice of a physical activity session that remedies the abovementioned drawbacks.

More specifically, the aim of the present invention is that of proposing an assistance device enabling a subject to carry out his/her exertion progressively, according to a predefined plan and under continuous automatic supervision of physiological parameters.

A further aim of the present invention is that of proposing a physical activity apparatus equipped with a device according to the invention.

Further aims and advantages of the present invention will emerge in the course of the description hereinafter which is merely given as an indication and is not intended to be limiting.

To this end, the invention relates to a device for assisting in the practice of a physical activity session by a subject, having electronic means for determining the intensity of the physical exertion, in real time, to be provided by said subject during said session, implementing a control law having, as an output, a parameter corresponding to said physical exertion to be provided, and, as input signals, data collected during the exertion, essentially consisting of:
the measurement of said heart rate of said subject,
the measurement of said respiratory rate of said subject,
one or a plurality of indices of strenuousness of the exertion perceived by said subject (i.e. general difficulty, dyspnoea, pain in the lower limbs), to be specified by said subject during the exertion,

and wherein the control law is also dependent on programmed thresholds, not to be exceeded, essentially consisting of:
the maximum heart rate,
the maximum respiratory rate,
the maximum value(s) associated with said index/indices of strenuousness of the exertion.

According to optional features taken along or in combination:
the device has means for inputting the values of the index/indices of strenuousness of the exertion comprising a touch screen or a microphone,
the device may have means for learning, during a prior session or during a first phase of the physical activity session, coefficients of the control law linking the parameter corresponding to the intensity of the physical exertion to be provided, the heart rate measured, the respiratory rate measured, and the index/indices of perceived strenuousness of the exertion;
the control law may be a robust control regulation law;
said device may have means for periodically transmitting physical activity instructions to maintain the physical exertion, increase the physical exertion or, on the contrary, decrease the physical exertion of the subject in correlation with the physical exertion to be provided by said subject, determined by said assistance device at the time preceding said physical activity instruction, and the actual exertion provided by said subject at the time preceding said physical activity instruction;
the device may have means for recording said programmed session data and the data collected during the session in a file.

The invention also relates to a physical activity apparatus equipped with an assistance device according to the invention.

According to optional features of said apparatus, taken alone or in combination:
the apparatus may have means for periodically adjusting the load of the physical activity apparatus in order to maintain the physical exertion, increase the physical exertion or, on the contrary, decrease the physical exertion of the subject in correlation with said physical exertion to be provided by said subject, determined by said assistance device at the time preceding the adjustment, and the actual exertion provided by said subject at the time preceding said adjustment;
the means for periodically adjusting the load of said apparatus may comprise a controlled brake (electromagnetic device or motor) or a variable-assist motor;
the actual exertion provided may be given by the measurement on the apparatus used for physical exercise of a resistance or assistance torque, work or power;
the apparatus may be selected from the following list: exercise bike, elliptical trainer, electric bike, treadmill, stepper, rowing machine, weight training bench.

The invention will be understood more clearly on reading the following description, accompanied by the appended figures, wherein:

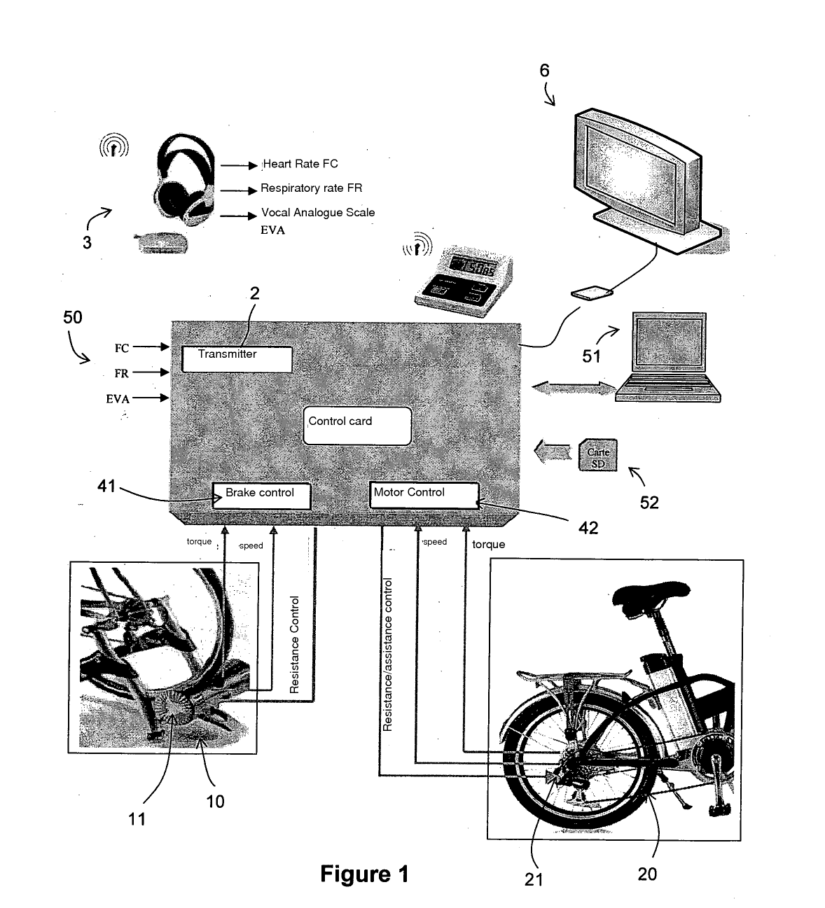

FIG. 1 schematically illustrates a physical activity apparatus equipped with an assistance device according to the invention.

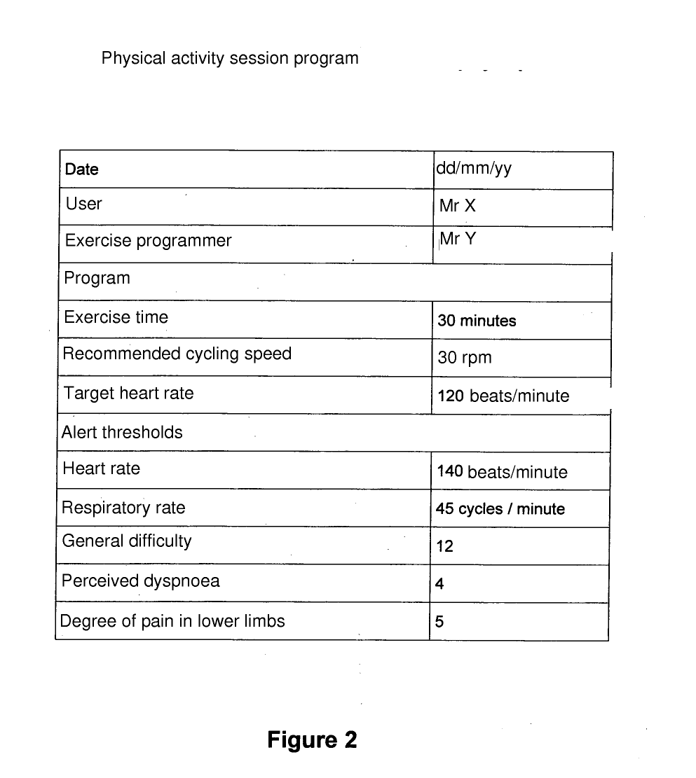

FIG. 2 is a table containing the programmed features of a physical activity session.

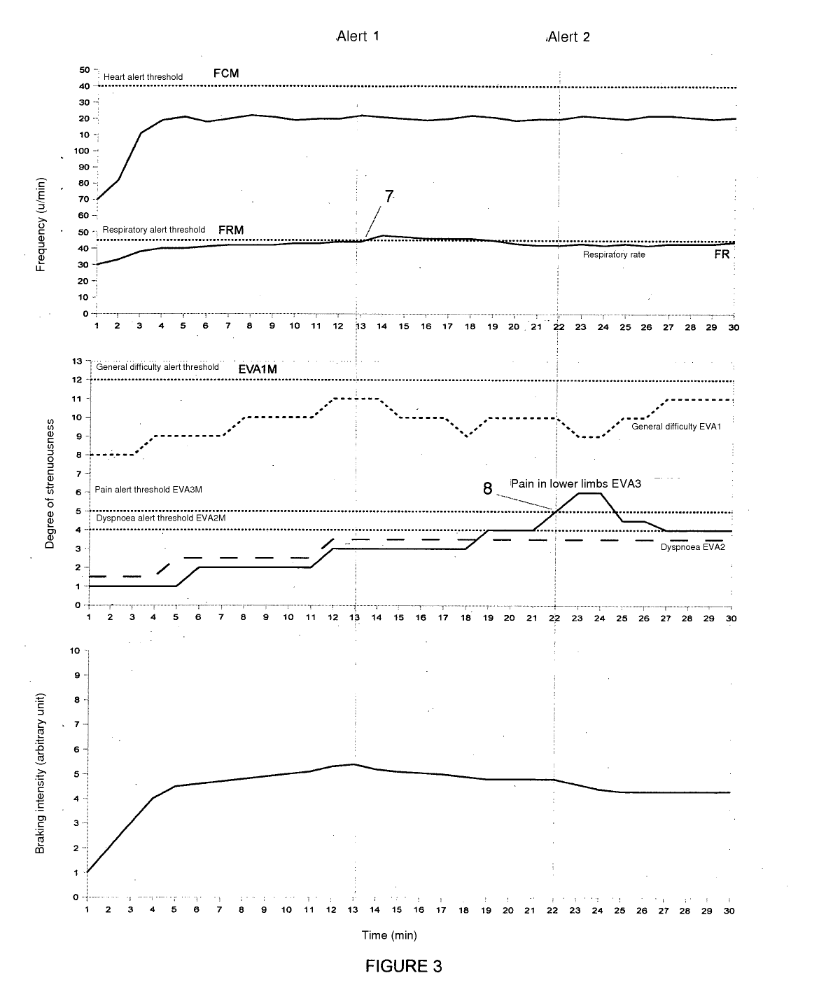

FIG. 3 is a multiple graph of the programmed session according to the features in the table in FIG. 2, with time on the X-axis, and respectively illustrating said heart rate measurement of said subject over time, the respiratory rate of said subject over time, the indices of strenuousness of the exertion perceived by said subject over time, and the physical exertion to be provided (on the assumption that the actual physical exertion corresponds to the physical exertion to be provided).

The invention relates to a device 1 for assisting in the practice of a physical activity session by a subject, having electronic means for determining the physical exertion, in real time, to be provided by said subject during said session, implementing a control law.

The control law has, as an output, a parameter corresponding to said physical exertion to be provided EPF, and, as input signals, data collected during the exertion, essentially consisting of:
the measurement of the heart rate FC of said subject,
the measurement of the respiratory rate FR of said subject,
one or a plurality of indices EVA of strenuousness of the exertion perceived by said subject, to be specified by said subject during the exertion, particularly measured using visual analogue scales and/or auditory analogue scales.

The invention comprises means for providing the values of the index/indices of strenuousness of the exertion which may comprise, for example, a touch screen or a microphone.

According to the non-limitative example in FIG. 3, there are three indices (EVA₁: general difficulty, EVA₂: dyspnoea, EVA₃: pain in the lower limbs).

The control law is also dependent on programmed thresholds, not to be exceeded, essentially consisting of:
the maximum heart rate FC_{M},
the maximum respiratory rate FR_{M},
the maximum value(s) EVA_{xM} associated with said index/indices of strenuousness of the exertion.

According to the non-limitative example in FIG. 3, there are three indices (EVA_{1M} for general difficulty, EVA_{2M} for dyspnoea, EVA_{3M} for pain in the lower limbs).

The control law thus makes it possible to define, periodically, for example every five seconds, said parameter corresponding to said physical exertion to be provided EPF such that each of the data collected complies with the programmed plan and remains below the danger thereof, in a stable manner, without intermittence.

To this end, advantageously, the control law may be a robust control regulation law. It particularly consists of regulation based on a control law using quasi-LPV or Takaji-Sugeno type models.

The control law may essentially consist of the following modelling equations:
a first equation linking:

the physical exertion to be provided and the heart rate

FC:EPF=f(FC)
a second equation lining the physical exertion to be provided EPF and the respiratory rate FR:

EPF=g(FR)
a third equation, or a series of equations, linking the physical exertion to be provided EPF and index or indices of strenuousness of the exertion perceived by said subject EVAₓ.

EPF=h(EVA)

A hierarchy is then established between these equations, for example: the first equation takes priority, ranking 1, the second equation ranks 2, and so on.

The first equation takes priority, the parameter corresponding to the physical exertion to be provided being determined on this basis at the start of the session, in relation with the heart rate.

However, once an input (the respiratory rate FR or one of the indices of perceived strenuousness of the exertion EVA) exceeds the associated threshold thereof (FR>FR_{M} or EVAₓ>EVA_{xM}), the equation corresponding to the input of the exceeded threshold takes priority.

For example:
FR>FR_{M}, the second equation takes priority and the parameter corresponding to the physical exertion to be provided is determined on this basis,
if EVAₓ>EVA_{xM}, the corresponding equation takes priority and the parameter corresponding to the physical exertion to be provided is determined on this basis.

According for the subject's age, weight and other physical criteria, the control law algorithm is specific to each subject.

According to one embodiment, the device may have means for learning, during a first phase of the physical activity session, coefficients of the control law linking the parameter corresponding to said physical exertion to be provided, the heart rate, the respiratory rate, and the index/indices of perceived strenuousness of the exertion.

The calculation of the coefficients may optionally be refined over time after said first phase.

According to a further embodiment, the learning may be carried out during a prior session, solely intended for this purpose.

The device may have means 2, 3 for periodically transmitting physical activity instructions to maintain the physical exertion, increase the physical exertion or, on the contrary, decrease the physical exertion of the subject in correlation with the physical exertion to be provided by said subject, determined by said assistance device at the time preceding said physical activity instruction, and the actual exertion provided by said subject at the time preceding said physical activity instruction;

For example, on the assumption that the heart rate is selected as the physical exertion parameter.

If said device determines the physical exertion to be provided EPF as 120 beats/min and the actual physical exertion is 115 beats/min, the device transmits an instruction to increase the physical exertion (increase speed, run faster, cycle faster, etc.).

If, on the other hand, the physical exertion is greater than 120 beats/min, such as for example 130 beats/min, the device transmits an instruction to decrease the physical exertion (reduce speed, run more slowly, cycle more slowly, etc.).

If the actual physical exertion corresponds to the physical exertion to be provided, according to a tolerance (+ or −4 beats/min) such as for example 114 beats/min, the instruction may be to maintain the speed.

The device may have means 51, 52 for recording said programmed session data and the data collected during the session in a file. The data collected are the heart rate FC, the respiratory rate FR, or the indices of the degree of strenuousness EVAₓ, periodically collected during the session.

A summary of the session is prepared and recorded automatically after calculating summary indices such as the maximum heart rate, the maximum respiratory rate, and means indices of strenuousness.

The programmed data may be the session duration, the desired physical exertion, the maximum heart rate FC_{M}, the maximum respiratory rate FR_{M}, and the maximum degree(s) of strenuousness of exertion EVA_{xM}, that have been programmed.

This personal record is used to monitor the subject's fitness and progress as the sessions are conducted. These data may be used and retained by a health professional.

The device may have means for displaying 6, in real time, said data collected during the exertion, or said programmed data. This screen displays the heart rate FC, the respiratory rate FR, or the indices of strenuousness EVAₓ of the exertion, in real time, during the session. These display means may be used to display the activity instructions which are displayed on the screen.

The invention also relates to a physical activity apparatus, equipped with an assistance device according to the invention. This apparatus may be selected from the following list: exercise bike 10, elliptical trainer, electric bike 20, treadmill, stepper, rowing machine, weight training bench.

That being said, the apparatus may have means 41; 42 for periodically adjusting the load of the physical activity apparatus 10; 20 in order to maintain the physical exertion, increase the physical exertion or, on the contrary, decrease the physical exertion of the subject in correlation with said physical exertion to be provided by said subject, determined by said assistance device at the time preceding the adjustment, and the actual exertion provided by said subject at the time preceding said adjustment.

For example on the assumption that the parameter corresponding to the physical exertion to be provided is the heart rate, let us assume that, at the time preceding the adjustment, the physical exertion to be provided determined by the assistance device 1 is 120 beats/min and that, at this time, the actual physical exertion provided by said subject is only 110 beats/min, the apparatus load will be increased by the means 41; 42, for example, by increasing the braking force of a controlled brake 11, or by decreasing the assistance provided by a variable-assist motor 21.

If, on the other hand, the actual exertion is greater than the physical exertion to be provided, determined by the assistance device, for example 130 beats/min, the adjustment will consist of decreasing the load of the physical activity apparatus by decreasing, for example, the braking force of a controlled brake 11, or by increasing the assistance provided by a variable-assist motor 21.

The physical activity apparatus according to the invention equipped with the assistance device 1 according to the example in FIG. 1 will now be described. This device comprises a box 50 incorporating electronics and a headset 3 equipped with a heart rate sensor, a respiratory rate sensor, a microphone, an earphone and a radiofrequency transmission/reception system (wireless transmission).

The heart rate sensor may be a commercial model with measurement on the earlobe or finger. The respiratory rate sensor, positioned in front of the mouth, may consist of a thermistor and electronics measuring the mean time interval between two successive temperature peaks or a microphone measuring breathing noise. The microphone may be a small commercial microphone connected to the transmitter/receiver.

The set of data collected, i.e. the heart rate FC, respiratory rate FR, the auditory signal, is transmitted via radiofrequency to the box 50 which has a transmitter/receiver for this purpose, such as an “MRF24J40MA 2.4 GHz microchip” in particular, coupled with a dsPic33 type microcontroller.

In this box, a microprocessor implementing the control law algorithm of the device is also programmed. The control law is a robust control regulation law using quasi-LPV or Takagi-Sugeno type models.

It may consist of the law developed above essentially consisting of a plurality of hierarchical equations with a change of hierarchy when an input (FR; FC; EVAₓ) exceeds the corresponding threshold thereof (FR_{M}, FC_{M}, EVA_{xM}).

This box is connected by a wired or wireless link to a computer 51 used to enter the programmed data, such as the session duration, desired activity intensity, particularly expressed in the form of work, and the thresholds, particularly the maximum heart rate FC_{M} not to be exceeded, the maximum respiratory rate FR_{M} not to be exceeded and the maximum index or indices of strenuousness of the exertion EVA_{xM} not to be exceeded. These data are sent to the box electronics.

During the session, the heart rate FC, the respiratory rate FR and the indices of perceived strenuousness of the exertion EVA are data compiled, in digital format, at regular intervals.

The box 50 may also be connected to a screen 6 via a video card and the connectors suitable for said screen. The transmitter/receiver 2 is also used for transmitting audio physical activity instructions, via the headset 3.

These instructions, as previously described (increase speed, reduce speed, maintain speed) are transmitted periodically to maintain the physical exertion or increase the exertion, or, on the contrary, decrease the physical exertion of the subject in correlation with the physical exertion to be provided by said subject, determined by the assistance device at the time preceding said physical activity instruction, and the actual exertion provided by said subject at the time preceding said physical activity instruction.

Alternatively or additionally, the apparatus may further have means 41; 42 for periodically adjusting the load of the physical activity apparatus 10; 20 in order to maintain the physical exertion, increase the physical exertion or, on the contrary, decrease the physical exertion of the subject in correlation with said physical exertion to be provided by said subject, determined by said assistance device at the time preceding the adjustment, and the actual exertion provided by said subject at the time preceding said adjustment.

The apparatus may be, as illustrated on the left part of FIG. 1, an exercise bike equipped with an Eddy current brake (electromagnetic brake) or a controlled torque electric motor acting as a brake. Alternatively, the device may be onboard an electric bike, the means for periodically adjusting the load comprising the variable-assist motor 21 of the electric bike.

If the actual exertion is less than the physical exertion to be provided EPF, determined by the device, the load adjustment consists of increasing the load, increasing the braking torque, or decreasing the assistance of the electric motor. If the actual exertion is greater than said exertion to be provided EPF, determined by the assistance device, the adjustment consists, on the other hand, of decreasing the braking torque or increasing the assistance of the variable-assist motor 21.

According to FIGS. 2 and 3, a physical activity session implemented by means of the assistance device 1 according to the invention will now be described.

As illustrated according to the table in FIG. 2, it consists of a programmed session having the following features:
duration 30 minutes,
desired physical exertion to be provided 120 beats/min,
maximum heart rate 140 beats/min,
maximum respiratory rate 45 cycles/min,
indices of maximum degree of strenuousness EVA_{1M}: 12 on a scale of 6 to 20 (general difficulty), EVA_{2M}: 4 on a scale of 0 to 10 (dyspnoea), EVA_{3M}: 5 on a scale of 0 to 10 (pain in lower limbs).

FIG. 3 illustrates the data collected during the session for a subject. The data illustrated are merely illustrative and do not reflect an actual scenario. The graph in FIG. 3 also illustrates the physical exertion to be provided determined by the device 1 during the session.

This example is based on the assumption that the actual exertion provided by the user corresponds to the physical exertion to be provided, determined by the device 1.

The data in FIG. 2 may be programmed by a health professional using the computer 51 and sent to the box 50 of the assistance device 1 according to the invention. In this example, the health professional decides that the user needs to complete 30 minutes of exertion of a constant heart rate of 120 beats/min. The respiratory rate should not exceed 45 breaths/minute and the heart rate should not exceed 140 beats/min. The user should not experience general difficulty greater than 12 on a scale of 6 to 20, a feeling of dyspnoea greater than 4 on a scale of 0 to 10, pain in the lower limbs greater than 5 on a scale of 0 to 10.

In view of the subject's age, weight and other physical criteria, the algorithm will first calculate an initial physical exertion value provided during the learning phase. During this phase, the measurements of the sensors (respiratory rate FR, heart rate FR and indices EVAₓ) will be used to calculate the coefficients of the control law equations (first equation, second equation, subsequent equations). More specifically, the heart rate sensor measures the heart rate. It may consist of a commercial model with measurement on the earlobe or finger.

The respiratory rate sensor may be positioned in front of the mouth, consist of a thermistor and electronic measuring the interval between two temperature peaks or a microphone and electronics measuring the breathing noise.

The indices of strenuousness of the exertion specified by the user during exertion may be given by voice instructions. The sensor may then consist of a microphone and electronics for translating the voice instructions into the values of the indices of strenuousness of the exertion (vocal analogue scale).

Alternatively, according to a further embodiment not shown, the indices of strenuousness of the exertion may be specified by the user by means of a touch screen displaying, for each index, a visual analogue scale, wherein each level corresponds to an index value.

The user specifies each index value by pressing the corresponding level on the visual scale.

The calculation may optionally be refined over time, after said first learning phase.

On the basis of the equations obtained, the model calculates the apparatus load required to achieve 120 beats/min. As illustrated, the heart rate increases progressively to achieve the desired target of 120 beats/min.

If, during the exertion, the respiratory rate exceeds 45 cycles/min as illustration according to the sampling referenced 7, the device 1 progressively reduces the physical exertion to be provided and thus the braking torque of the controlled brake 11 (or, on the contrary, increases the assistance of the motor 21) until the respiratory rate returns to less than or equal to the maximum authorised respiratory rate.

During the exertion, the subject is invited to specify the indices of perceived strenuousness of the exertion. If, for example, one of these indices is greater than or equal to the maximum authorised pain, the device decreases the physical exertion to be provided, for example, by decreasing the braking torque or by increasing the assistance of the variable-assist electric motor 21.

In FIG. 3, it can be seen that the threshold EVA_{3M} (pain in lower limbs) is exceeded, at the point referenced 8.

In any case, the control law observes the respective programmed thresholds not to be exceeded, and corrects the physical exertion to be provided so as to observe said thresholds.

Naturally, further embodiments could be envisaged by those skilled in the art without leaving the scope of the claims defined hereinafter.

1. Device (1) for assisting in the practice of a physical activity session by a subject, having electronic means for determining the intensity of the physical exertion, in real time, to be provided by said subject during said session, implementing a control law having, as an output, a parameter corresponding to said physical exertion to be provided (EPF), and, as input signals, data collected during the exertion, essentially consisting of:
the measurement of said heart rate (FC) of said subject,
the measurement of said respiratory rate (FR) of said subject,
one or a plurality of indices (EVAₓ) of strenuousness of the exertion perceived by said subject, to be specified by said subject during the exertion,
and wherein the control law is also dependent on programmed thresholds, not to be exceeded, essentially consisting of:
the maximum heart rate FC_{M},
the maximum respiratory rate FR_{M},
the maximum value(s) EVA_{xM} associated with said index/indices of strenuousness of the exertion.

2. Device according to claim 1, having means for inputting the values of the index/indices of strenuousness of the exertion comprising a touch screen or a microphone.

3. Device according to claim 1, having means for learning, during a prior session or during a first phase of the physical activity session, coefficients of the control law linking the parameter corresponding to the intensity of the physical exertion to be provided, the heart rate measured, the respiratory rate measured, and the index/indices of perceived strenuousness of the exertion0

4. Device according to claim 1, wherein the control law is a robust control regulation law.

5. Device according to claim 1, wherein said device has means (2, 3) for periodically transmitting physical activity instructions to maintain the physical exertion, increase the physical exertion or, on the contrary, decrease the physical exertion of the subject in correlation with the physical exertion to be provided by said subject, determined by said assistance device at the time preceding said physical activity instruction, and the actual exertion provided by said subject at the time preceding said physical activity instruction.

6. Device according to claim 1, having means (51; 52) for recording said programmed session data and the data collected during said session with a summary in a file.

7. Device according to claim 1 any of claims 1 to 6, having means for displaying (6), in real time, said data collected during the exertion and said programmed data.

8. Physical activity apparatus (10; 20) equipped with an assistance device according to claim 1.

9. Apparatus according to claim 8, having means (41; 42) for periodically adjusting the load of the physical activity apparatus (10; 20) in order to maintain the physical exertion, increase the physical exertion or, on the contrary, decrease the physical exertion of the subject in correlation with said physical exertion to be provided by said subject, determined by said assistance device at the time preceding the adjustment, and the actual exertion provided by said subject at the time preceding said adjustment.

10. Apparatus according to claim 9, wherein said means (41; 42) for periodically adjusting the load of said apparatus comprise a controlled brake (11) or a variable-assist motor (21).

11. Apparatus according to claim 8, wherein the actual exertion provided is given by the measurement on the apparatus used for physical exercise of a resistance or assistance torque, work or power.

12. Apparatus according to claim 8, wherein said apparatus is selected from the following list: exercise bike (10), elliptical trainer, electric bike (20), treadmill, stepper, rowing machine, weight training bench.

13. Device according to claim 2, having means for learning, during a prior session or during a first phase of the physical activity session, coefficients of the control law linking the parameter corresponding to the intensity of the physical exertion to be provided, the heart rate measured, the respiratory rate measured, and the index/indices of perceived strenuousness of the exertion0

14. Device according to claim 2, wherein the control law is a robust control regulation law.

15. Device according to claim 2, wherein said device has means (2, 3) for periodically transmitting physical activity instructions to maintain the physical exertion, increase the physical exertion or, on the contrary, decrease the physical exertion of the subject in correlation with the physical exertion to be provided by said subject, determined by said assistance device at the time preceding said physical activity instruction, and the actual exertion provided by said subject at the time preceding said physical activity instruction.

16. Device according to claim 2, having means (51; 52) for recording said programmed session data and the data collected during said session with a summary in a file.

17. Device according to claim 2 any of claims 1 to 6, having means for displaying (6), in real time, said data collected during the exertion and said programmed data.
